(19) United States
(12) Patent Application Publication
(10) Pub. No.: US 2025/0057992 A1
(43) Pub. Date: Feb. 20, 2025

(54) MICROFLUIDIC PREPARATION OF NANODROPLETS WITH FLUORINATED COMPOUND

(71) Applicant: Bracco Suisse SA, Cadempino (CH)

(72) Inventors: Romain MELICH, Choisy (FR); Philippe BUSSAT, Pers-Jussy (FR); David LAZARUS, Saint-Julien-en-Genevois (FR); Samir CHERKAOUI, Feigeres (FR)

(73) Assignee: Bracco Suisse SA, Cadempino (CH)

(21) Appl. No.: 18/722,126

(22) PCT Filed: Dec. 21, 2022
(86) PCT No.: PCT/EP2022/087376

§ 371 Date: Jun. 20, 2024

(30) Foreign Application Priority Data
Dec. 22, 2021 (EP) 21217022.9

(51) Int. Cl. A61K 49/22; A61N 7/00

(52) U.S. Cl. CPC A61K 49/223; A61K 49/226; A61N 7/00; A61N 2007/0039

(57) ABSTRACT

Method for the preparation of an aqueous suspension of calibrated nanodroplets with a core comprising a low boiling fluorinated compound through microfluidic technology.

### TECHNICAL FIELD

The invention generally relates to a method for the preparation of an aqueous suspension of calibrated nanodroplets with a core comprising a low boiling fluorinated compound through microfluidic technology. The invention further relates to an aqueous suspension comprising a plurality of said nanodroplets obtained by said method, for use in a diagnostic and/or therapeutic treatment.

### BACKGROUND OF THE INVENTION

Phase-change contrast agents (PCCAs), also known as acoustically activated nanodroplets, are receiving increased popularity in both ultrasound diagnostic and therapeutic delivery. Except for the core, often consisting of liquid perfluorocarbons, nanodroplets display similar composition to commercially available gas-filled microbubbles. Owing to Acoustic Droplet Vaporization (ADV) process, encapsulated droplets are converted into gas bubbles upon exposure to ultrasound energy beyond a vaporization threshold. In fact, ultrasounds act as a remote trigger to promote the vaporization of the droplets in a controllable, non-invasive and localized manner. Thanks to their liquid core and smaller size compared to conventional microbubbles, nanodroplets display prolonged in vivo circulation and deep penetration into the tissues via the extravascular space. Moreover, below vaporization threshold, they are ultrasonically stable with low acoustic attenuation and can be acoustically vaporized at the location of interest.

Perfluorocarbon nanodroplets present a real potential as an extravascular ultrasound contrast agent in numerous diagnostic and therapeutic applications including sonopermeabilization, blood brain barrier (BBB) disruption, multimodal imaging modalities and to allow passive (due to the enhanced permeability and retention (EPR) effect in the tumor tissues) or active targeting (by incorporating targeted ligands) for localized delivery of therapeutic drugs or genes. Another potentially valuable characteristic of (P)FC-NDs is their possible application for novel imaging strategies such as UltraSound Super-Resolution Imaging since these agents can be activated and deactivated on demand by applying intermittent acoustic pulses.

PCCAs are usually composed by an outer stabilizing shell, consisting of lipids, surfactants, polymers or proteins, and an inner core comprising a fluorinated compound. Depending on the encapsulated fluorinated compound, it is possible to distinguish between PCCAs comprising fluorinated compound with boiling points above room temperature (RT; 25° C.), i.e. fluorinated compounds which are in a liquid form at Standard Ambient Temperature and Pressure (SATP), namely at 25° C. and 1 atm (101.325 kPa), and low boiling PCCAs, comprising fluorinated compounds with boiling points below room temperature, i.e. (P)FCs which are a gas at SATP.

Recently, low boiling PCCAs have attracted much attention due to their intrinsic characteristics. Once prepared, low boiling nanodroplets remain metastable in liquid form even at temperatures well above the bulk boiling point of the core comprising a fluorinated compound, due to homogenous nucleation and fluorinated compound intermolecular forces. For this reason, low boiling nanodroplets can undergo ADV at much lower acoustic pressures than those required to vaporize liquid PCCAs, enabling their use in a wider variety of biomedical applications, reducing negative bioeffects related to the use of high activation energy (Durham and Dayton, 2021).

As with liquid PCCAs, the preparation of monodispersed low boiling PCCAs represents an area of growing interest due to their advantages, such a higher vaporization efficiency compared to broadly distributed nanodroplets.

Sheeran, 2011, describes the preparation of lipid-encapsulated droplets comprising decafluorobutane (DFB; boiling point −2° C.) through two different methods. The former involves condensing DFB gas followed by extrusion with a lipid formulation in a buffer; the latter is based on generating lipid-coated nanodroplets through pressurization and condensation of preformed microbubbles containing DFB.

US2019/0307908 proposes a method to produce PFC-NDs, including PFCs with boiling points ranging from −37 to 56° C., based on the spontaneous nucleation, referred to as the ouzo method. The process relies on saturating a cosolvent with the PFC before adding a poor solvent to reduce solvent quality, forcing droplets to spontaneously nucleate.

Recently variants of the flow-focusing microfluidics technique have been investigated to generate uniformly sized liquid perfluorocarbon PCCAs (Durham and Dayton 2021). Moreover, a new microfluidic approach based on a new mixing system to enhance the mixing of two phases has been proposed for the preparation of liquid perfluorocarbon PCCAs (Melich, 2020).

However, the severe working conditions that low-boiling point PCCAs require for their handling, such as operating at relatively low temperatures, represent a challenging area for the manufacturing of the low-boiling point PCCAs though microfluidic technique.

Up to now, to the best of Applicant's knowledge, the microfluidic technique has not yet been applied for the preparation of monodisperse low boiling-point nanodroplets. As used herein, the term “monodisperse” (or “calibrated”) refers to a population of particles having a relatively low size distribution around a mean value, as defined in detail in the following paragraphs of this description.

The Applicant is now disclosing a microfluidic approach to prepare calibrated low-boiling PCCAs that aims at overcoming the technical issues traditionally related to the use of this technique, such as managing cryogenic conditions.

### SUMMARY OF THE INVENTION

A first aspect of the invention relates to a method for the preparation of an aqueous suspension of calibrated nanodroplets, said nanodroplets comprising an outer shell and an inner core, wherein said outer shell comprises an amphiphilic component and said inner core comprises a low boiling fluorinated compound, said method comprising:
a) Preparing an aqueous phase;
b) Preparing an organic phase; wherein
   said aqueous phase comprises an amphiphilic component and the organic phase comprises a low boiling fluorinated compound or
   said organic phase comprises an amphiphilic component and a low boiling fluorinated compound;
c) Injecting said aqueous phase into a first inlet and said organic phase into a second inlet of a microfluidic cartridge thereby mixing said aqueous phase and said organic phase in a mixing device of the microfluidic cartridge to obtain an aqueous suspension of calibrated nanodroplets; and
d) Collecting the aqueous suspension of calibrated nanodroplets from the exit channel of the microfluidic cartridge.

In a preferred embodiment, the organic phase comprises an amphiphilic component and a low boiling fluorinated compound.

In another embodiment said low boiling fluorinated compound has a boiling point lower than 25° C.

In a further embodiment, said low boiling fluorinated compound is preferably a low boiling perfluorocarbon.

According to another embodiment, said step b) relates to a method for the preparation of an organic phase comprising a low boiling fluorinated compound, said method comprising the steps of:
b.1) Condensing a gaseous low boiling fluorinated compound to obtain a liquid low boiling fluorinated compound;
b.2) Preparing a liquid organic solution comprising an organic solvent;
b.3) Cooling said liquid organic solution down to a temperature below the boiling point of said liquid low boiling fluorinated compound;
b.4) Mixing said liquid low boiling fluorinated compound and said liquid organic solution to obtain an organic phase.

According to a preferred embodiment, step b.1) is performed by cooling said gaseous low boiling fluorinated compound down to a temperature below the boiling point of said low boiling fluorinated compound.

In another embodiment, at step b.4) the temperature at which the mixing is performed is a temperature below the boiling point of said low boiling fluorinated compound.

Preferably, the temperature at step b.4) is the same temperature of step b.3).

In a preferred embodiment, the temperature of the obtained organic phase at the end of step b.4) is lower than the boiling point of said low boiling fluorinated compound, preferably is at least 5° C. lower than the boiling point of said low boiling fluorinated compound, more preferably at least 10° C. lower, even more preferably at least 20° cs lower, up to e.g. 50° C. lower.

In an alternative embodiment, said step b) relates to a method for the preparation of an organic phase comprising a low boiling fluorinated compound, said method comprising the steps of:
b.i) Preparing a liquid organic solution comprising an organic solvent, and
b.ii) Admixing a gaseous low boiling fluorinated compound to said organic liquid solution.

Preferably at step b.ii) the admixing is performed by bubbling the gaseous low boiling fluorinated compound into the organic solution.

According to a preferred embodiment, before the admixing of step b.ii) the temperature of the organic solution is above the boiling point of said gaseous low boiling fluorinated compound.

Preferably, said temperature is between the boiling point and room temperature (RT), more preferably up to 15° C. higher than the boiling point, even more preferably up to 10° C. higher.

Another aspect of the invention relates to an aqueous suspension comprising a plurality of calibrated nanodroplets, said nanodroplets comprising an outer shell and an inner core, wherein said outer shell comprises an amphiphilic component and said inner core comprises a low boiling fluorinated compound, said suspension obtainable by the process as defined above.

A further aspect relates to an aqueous suspension comprising a plurality of calibrated nanodroplets, said nanodroplets comprising an outer shell and an inner core, wherein said outer shell comprises an amphiphilic component and said inner core comprises a low boiling fluorinated compound, obtained according to the process as above defined for use in a diagnostic and/or therapeutic treatment

### FIGURES

FIG. 1 is a schematic representation of the core portion of a microfluidic cartridge.

FIG. 2 shows a schematic representation of a cross-section of a staggered herringbone mixer (SHM) design.

### DETAILED DESCRIPTION OF THE INVENTION

The present invention generally relates to a microfluidic method for the preparation of a composition comprising calibrated nanodroplets, wherein said nanodroplets comprise an outer shell and an inner core, wherein said outer shell comprises an amphiphilic component and said inner core comprises a low boiling fluorinated compound.

Said calibrated nanodroplets are suitable as contrast agents in ultrasound imaging techniques, known as Contrast-Enhanced Ultrasound (CEUS) Imaging, or in therapeutic applications, e.g. thermal ablation or for ultrasound mediated drug/gene delivery.

### Definitions

The expression “(low boiling (P)FC-ND)” indicates an assembly comprising an outer layer and an inner core, said outer layer comprising an amphiphilic component and said inner core comprising a liquid low boiling fluorinated compound.

The expression “low boiling fluorinated compound” indicates a highly volatile fluorinated compound, in particular a gas having a boiling point lower than 25° C., i.e. characterized by being a gas at Standard Ambient Temperature and Pressure (SATP), namely at 25° C. and 1 atm (101.325 kPa).

In this description the expression “fluorocarbon” indicates a fluorinated compound, such as hydrofluorocarbons, both saturated and unsaturated, perfluorocarbons, fluorinated ethers, fluorinated ketones or perfluorinated nitrile.

Said nanodroplets are commonly referred to as “phase change contrast agents”, since they can be activated by an external stimulus to convert from a liquid state to a gas state. In particular, the liquid low boiling fluorinated compound within the droplet (which is in a metastable liquid state at room temperature or when administered into a body) can undergo to a phase-change becoming a gas when submitted to an external stimulus, such as ultrasonic, X-ray, optical, infrared, microwave or radio frequency energy.

The expression “acoustic droplet vaporization” (ADV) refers to the phenomenon wherein said low boiling (P)FC-NDs can be converted in bubbles by exposure to ultrasonic energy, with the corresponding increase in size, e.g. from nanometric to larger size echogenic bubbles.

The term “bubble” as used herein refers to an assembly comprising an outer layer and an inner core, said outer layer comprising an amphiphilic component and said inner core comprising a gaseous low boiling fluorinated compound. Preferably said bubbles are microbubbles.

Due to their nanometric sizes, after in vivo administration said low boiling (P)FC nanodroplets can extravasate, for instance into the interstitial space of a solid tumor, and provide sufficient contrast for ultrasound imaging after their conversion into bubbles upon exposure to ultrasound energy beyond a vaporization threshold.

Preferably, for the extravasation the mean diameter of said low boiling (P)FC nanodroplets is lower than 400 nm, e.g. between 100 nm and 300 nm.

Said low boiling (P)FC nanodroplets are referred to as “metastable”, because they are stable as droplets at room conditions (i.e. SATP) and physiological conditions (typically a temperature from 36.5 to 37.5° C. and a pressure of 120/80 mm/Hg), meaning that they do not spontaneously expand into gas bubbles without being submitted to an external acoustic energy. For instance, after their administration, the sole exposition of said low boiling (P)FC-NDs to the body temperature and physiological pressure do not cause their activation and conversion in microbubbles. Additional energy is thus required to trigger this phenomenon after administration, such as ultrasound stimulus provided by a medical device.

The presence of a low boiling fluorinated compound in the inner core makes the resulting droplets acoustically activatable with substantially less energy than other phase-change contrast agents, e.g. similarly-sized nanodroplets comprising higher boiling point PFCs, endowing to considerable advantages for their applications in diagnostics, therapeutics and other treatments.

The expression “calibrated” (or “monodisperse”) refers to a population of nanodroplets as above defined, wherein said vesicles have a z-average diameter comprised between 100 nm and 1000 nm and a polydispersity lower than 0.20.

The expression “calibrated nanodroplets” indicates a plurality of calibrated nanodroplets as above defined typically dispersed or suspended in an aqueous carrier, preferably obtained through microfluidic technique.

### Amphiphilic Components

In the present description and claims, the expression “amphiphilic component” has its conventional meaning in the chemical field and refers to an organic compound, comprising a hydrophilic moiety and a hydrophobic moiety, suitable for forming the stabilizing layer (i.e. outer layer) of the nanodroplets. In said nanodroplet, the amphiphilic component molecules are oriented in such a way that the hydrophobic portions are located at the surface of the liquid low boiling point fluorinated compounds placed in the inner core.

The disclosed low boiling (P)FC-NDs are generally stabilized by one or more amphiphilic component. Suitable amphiphilic components comprise, for instance, fluorinated surfactants; phospholipids; lysophospholipids; fatty acids, such as palmitic acid, stearic acid, arachidonic acid or oleic acid; lipids bearing polymers, such as chitin, hyaluronic acid, polyvinylpyrrolidone or polyethylene glycol (PEG), also referred as “pegylated lipids”; lipids bearing sulfonated mono- di-, oligo- or polysaccharides; cholesterol, cholesterol sulfate or cholesterol hemisuccinate; tocopherol hemisuccinate; lipids with ether or ester-linked fatty acids; polymerized lipids; diacetyl phosphate; dicetyl phosphate; ceramides; polyoxyethylene fatty acid esters (such as polyoxyethylene fatty acid stearates), polyoxyethylene fatty alcohols, polyoxyethylene fatty alcohol ethers, polyoxyethylated sorbitan fatty acid esters, ethylene oxide (EO) and propylene oxide (PO) block copolymers (Pluronic or Poloxamer); esters of sugars with aliphatic acids including sucrose laurate, fructose laurate, sucrose palmitate, sucrose stearate, glycerol or glycerol monoesters with fatty acids, including glycerol monopalmitate, glycerol monostearate, glycerol monomyristate or glycerol monolaurate; long chain alcohols including n-decyl alcohol, lauryl alcohol, myristyl alcohol, cetyl alcohol, or n-octadecyl alcohol; 1,2-dioleyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine; alkylamines or alkylammonium salts, comprising at least one (C₁₀-C₂₀), preferably (C₁₄-C₁₈), alkyl chain, such as, for instance, N-stearylamine, N,N′-distearylamine, N-hexadecylamine, N,N′-dihexadecylamine, N-stearylammonium chloride, N,N′-distearylammonium chloride, N-hexadecylammonium chloride, N,N′-dihexadecylammonium chloride, dimethyldioctadecylammonium bromide (DDAB), hexadecyltrimethylammonium bromide (CTAB); tertiary or quaternary ammonium salts comprising one or preferably two (C₁₀-C₂₀), preferably (C₁₄-C₁₈), acyl chain linked to the N-atom through a (C₃-C₆) alkylene bridge, such as, for instance, 1,2-distearoyl-3-trimethylammonium-propane (DSTAP), 1,2-dipalmitoyl-3-trimethylammonium-propane (DPTAP), 1,2-oleoyl-3-trimethylammonium-propane (DOTAP), 1,2-distearoyl-3-dimethylammonium-propane (DSDAP),(N-(4-carboxybenzyl)-N,N-dimethyl-2,3-bis(oleoyloxy)propan-1-aminium (DOPAQ), (6Z,9Z,28Z,31Z)-Heptatriaconta-6,9,28,31-tetraen-19-yl 4-(dimethylamino)butanoate (DLin-MC3-DMA) or 2-[2,2-bis[(9Z,12Z)-octadeca-9,12-dienyl]-1,3-dioxolan-4-yl]-N,N-dimethylethanamine (DLin-KC2-DMA); and mixtures or combinations thereof.

According to an embodiment, the amphiphilic component is a fluorinated surfactant.

In the present description and claims, the expression “fluorinated surfactant” refers to an amphiphilic organic compound suitable for forming the stabilizing layer of the nanodroplets, comprising a hydrophilic moiety and a hydrophobic moiety, said hydrophobic moiety comprising fluorine atoms (i.e. a fluorocarbon part). Said fluorinated surfactant advantageously exhibits a high affinity for both the inner core, comprising a low boiling fluorinated compound and the surrounding water, being the nanodroplets preferably dispersed in an aqueous solvent.

Examples of suitable fluorinated surfactants include non-ionic linear fluorosurfactants, such as the non-ionic linear ethoxylated fluorosurfactant Zonyl FSO, or fluorinated polymers, such as fluorinated aminoacid sequences.

According to a preferred embodiment, said fluorinated surfactant is Zonyl FSO.

According to a further embodiment, the amphiphilic component is a phospholipid.

The term “phospholipid” is intended to encompass any amphiphilic phospholipidic compound, the molecules of which can form a stabilizing film of material at the fluorinated compound-aqueous phase boundary interface in the final calibrated low boiling (P)FC-NDs suspension.

Examples of suitable phospholipids include esters of glycerol with one or preferably two (equal or different) residues of fatty acids and with phosphoric acid, wherein the phosphoric acid residue is in turn bound to a hydrophilic group, such as, for instance, choline (phosphatidylcholines—PC), serine (phosphatidylserines—PS), glycerol (phosphatidylglycerols—PG), ethanolamine (phosphatidylethanolamines—PE), inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing from 12 to 24 carbon atoms, preferably from 14 to 22; the aliphatic chain may contain one or more unsaturations or is preferably completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Preferably, saturated fatty acids such as myristic acid, palmitic acid, stearic acid and arachidic acid are employed.

Further examples of phospholipids are phosphatidic acids, i.e. the diesters of glycerol-phosphoric acid with fatty acids; sphingolipids such as sphingomyelins, i.e. those phosphatidylcholine analogues where the residue of glycerol diester with fatty acids is replaced by a ceramide chain; cardiolipins, i.e. the esters of 1,3-diphosphatidylglycerol with a fatty acid; glycolipids such as gangliosides GM1 (or GM2) or cerebrosides; glucolipids; sulfatides and glycosphingolipids.

As used herein, the term “phospholipids” include either naturally occurring, semisynthetic or synthetically prepared products that can be employed either singularly or as mixtures.

Examples of naturally occurring phospholipids are natural lecithins (phosphatidylcholine (PC) derivatives) such as, typically, soya bean or egg yolk lecithins.

Examples of semisynthetic phospholipids are the partially or fully hydrogenated derivatives of the naturally occurring lecithins. Preferred phospholipids are fatty acids diesters of phosphatidylcholine, ethylphosphatidylcholine, phosphatidylglycerol, phosphatidic acid, phosphatidylethanolamine, phosphatidylserine, phosphatidylinositol or of sphingomyelin.

Examples of preferred phospholipids are, for instance, dilauroylphosphatidylcholine (DLPC), dimyristoylphosphatidylcholine (DMPC), dipalmitoylphosphatidylcholine (DPPC), diarachidoylphosphatidylcholine (DAPC), distearoylphosphatidylcholine (DSPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroyl-phosphatidylglycerol (DLPG) and its alkali metal salts, diarachidoylphosphatidyl-glycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid (DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoyl-phosphatidylethanolamine (DMPE), dipalmitoylphosphatidylethanolamine (DPPE), distearoylphosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoylphosphatidylserine (DMPS), diarachidoyl phosphatidylserine (DAPS), dipalmitoylphosphatidylserine (DPPS), distearoylphosphatidylserine (DSPS), dioleoylphosphatidylserine (DOPS), dipalmitoylsphingomyelin (DPSP), distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoyl phosphatidylinositol (DSPI), dioleoyl-phosphatidylinositol (DOPI).

Suitable phospholipids further include phospholipids modified by linking a hydrophilic polymer, such as polyethyleneglycol (PEG) or polypropyleneglycol (PPG), thereto. Preferred polymer-modified phospholipids include “pegylated phospholipids”, i.e. phospholipids bound to a PEG polymer. Examples of pegylated phospholipids are pegylated phosphatidylethanolamines (“PE-PEGs” in brief) i.e. phosphatidylethanolamines where the hydrophilic ethanolamine moiety is linked to a PEG molecule of variable molecular weight (e.g. from 300 to 20000 daltons, preferably from 500 to 5000 daltons), such as DPPE-PEG (or DSPE-PEG, DMPE-PEG, DAPE-PEG or DOPE-PEG). For example, DPPE-PEG2000 refers to DPPE having attached thereto a PEG polymer having a mean average molecular weight of about 2000.

Mixtures of phospholipids can also be used, such as, for instance, mixtures of DPPE and/or DSPE (including pegylated derivatives), DPPC, with DMPA, DPPA, DPPG, or Ethyl-DPPC.

For instance, a mixture of phospholipids may include phosphatidylcholine derivatives, phosphatidic acid derivatives and pegylated phosphatidylethanolamine, e.g. DPPC/DMPA/DPPE-PEG, DPPC/DPPA/DSPE-PEG, DPPC/DPPG/DPPE-PEG, DPPC/DPPG/DSPE-PEG.

According to the present invention, the phospholipid can conveniently be used in admixtures with any of the above listed amphiphilic compounds. Thus, for instance, lipids such as cholesterol, ergosterol, phytosterol, sitosterol, lanosterol, tocopherol, propyl gallate or ascorbyl palmitate, fatty acids such as myristic acid, palmitic acid, stearic acid, arachidic acid and derivatives thereof or butylated hydroxytoluene and/or other non-phospholipid compounds can optionally be added to one or more of the foregoing phospholipids. For instance, mixtures of amphiphilic materials comprising phospholipids and fatty acids can advantageously be used, including DPPC/DPPE-PEG/palmitic acid, DPPC/DSPE-PEG/palmitic acid, DPPC/DPPE-PEG/stearic acid, or DPPC/DSPE-PEG/stearic acid.

According to an embodiment, the outer layer of said calibrated low boiling (P)FC-NDs comprises a mixture of amphiphilic components. Preferably said mixture comprises phospholipids and a lipid bearing polymer.

According to a preferred embodiment, said lipid bearing polymers is a pegylated phospholipid, as defined above.

According to a further preferred embodiment, said mixture further comprises a fatty acid, preferred being palmitic acid.

The Applicant observed that the presence of a fatty acid (e.g. palmitic acid) in the formulation played a role in controlling sizes, PDI and stability of the calibrated low boiling (P)FC-NDs. In particular, compositions not comprising a fatty acid, displayed higher NDs sizes in comparison with the other formulations having increasing amount of palmitic acid.

Preferably the concentration of fatty acid, and particularly of palmitic acid, in said mixture of amphiphilic components is comprised between 1 and 80%, preferably between 10 and 60%, still more preferably between 15 and 55%.

### Fluorinated Compounds

In the present description and claims the term “fluorinated compound” refers to a group of fluorine-containing compounds derived from (optionally substituted) hydrocarbons by partial or complete substitution of hydrogen atoms with fluorine atoms, which are gas at SATP conditions (i.e. highly volatile fluorinated compounds). Suitable examples of fluorinated compounds are hydrofluorocarbons, both saturated and unsaturated, perfluorocarbons, fluorinated ethers, fluorinated ketones or perfluorinated nitrile. Preferably the fluorinated compound is a perfluorocarbon (PFC), i.e. a fluorinated hydrocarbon where all the hydrogen atoms are substituted with fluorine atoms.

Gaseous fluorinated compounds are characterized by a boiling point comprised between −70° C. and 25° C., at atmospheric pressure. In the present invention, the fluorinated compounds are preferably characterized by a boiling point comprised between −50° C. and 22° C., more preferably between −45° C. and 18° C., still more preferably between −40° C. and 5° C.

Suitable examples of fluorinated compounds include C₃-C₄ fluorinate compounds, such as 1,1,1,2,3,3,3 heptafluoropropane, 1,1,1,2,2,3-Hexafluoropropane, 1,1,1,2,3,3-Hexafluoropropane, 1,1,1,3,3,3-Hexafluoropropane, 1,1,1,2,2,3,3,4,4 Nonafluorobutane, 1,1,1,3,3,3-Hexafluoro-2-(trifluoromethyl)propane, 1,1,1,2,2,3,3,4 Octafluorobutane or a mixture thereof.

Suitable examples of perfluorocarbons are perfluorocyclopropane, perfluoropropane, perfluorocyclobutane, perfluorobutane, perfluoroisobutane or a mixture thereof.

In an embodiment said perfluorocarbon is preferably perfluorobutane (boiling point −2° C.) or perfluoropropane (boiling point −37° C.).

### Microfluidic Cartridge

The calibrated low boiling (P)FC-NDs of the present invention are preferably produced through a bottom-up approach using a microfluidic technique.

In the present description and claims the expression “microfluidic technique” refers to a technology of manufacturing nanodroplets through a microfluidic cartridge designed to manipulate fluids in channels at the microscale.

Said microfluidic technique is a bottom-up approach, that is to say that the nanodroplets are obtained by assembling molecules (e.g. amphiphilic components and fluorinated compounds) into larger nanostructures (i.e. nanodroplets).

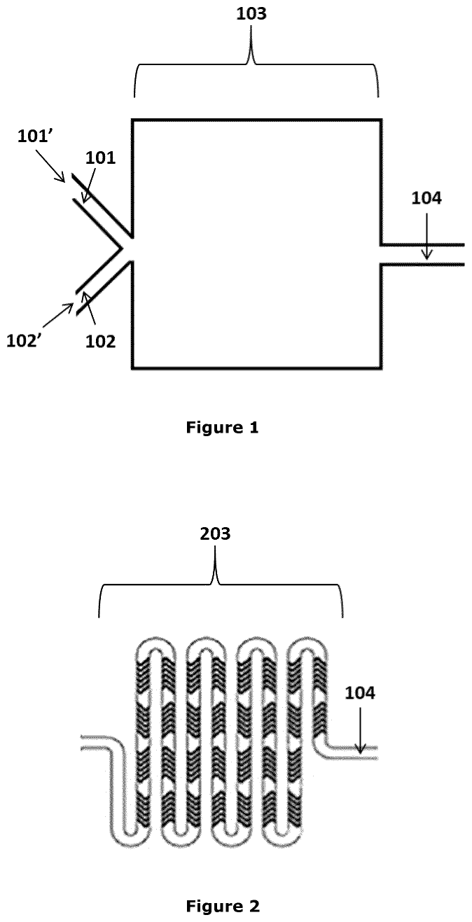

FIG. 1 shows a schematic representation of the core portion 100 of a microfluidic cartridge (i.e. microfluidic cartridge) useful in the process of the invention. The cartridge comprises a first inlet 101 for feeding the aqueous phase 101′ and a second inlet 102 for supplying the organic phase 102′. The aqueous phase and the organic phase are directed towards a mixing device 103, for instance a staggered herringbone micromixer 203 as illustrated in FIG. 2, wherein they are mixed (e.g. through laminar mixing in the case of the micromixer of FIG. 2 endowing to the formation of NDs.

The calibrated NDs are then directed to the exit channel 104, from where they are collected in a suitable container (e.g. a vial).

Alternatively, said microfluidic cartridge can be equipped with an additional channel, for instance placed between the mixing device 103 and the exit channel 104, aimed at diluting, with a suitable solvent, the calibrated NDs suspension before their direction to the exit channel 104 (i.e. in-line dilution).

A mixing device 103 is generally characterized by suitable geometries able to enhance the microfluidic-mixing performance. In fact, the mixing process takes place into the peculiar micro-channel geometry of the mixing device, which causes fluid streams to mix together on the way to exit the microfluidic cartridge.

Different types of mixing devices are available with different shapes or microstructures. Suitable examples of mixing devices can be classified as passive micromixers, such as T and Y shaped mixers (e.g. staggered herringbone micromixer or toroidal mixer), and the mixer using flow focusing; and active micromixers, such as mixer using pressure field disturbance, electrokinetic active micromixer and ultrasound active micromixer.

Preferred in the present invention is a staggered herringbone micromixer (FIG. 2), wherein the mixing of the two liquid phases is controlled by lamination-mixing or a toroidal micromixer.

During the mixing phase the nanodroplets are formed and directed to the exit channel of the microfluidic cartridge, or, alternatively, directed to an additional channel to dilute the nanodroplets before their direction to the exit channel.

In the present description and claims, the expression “exit (or outlet) channel” indicates the terminal portion of the microfluidic cartridge, toward which the just formed nanodroplets are directed from the mixing device and from where it is possible to collect the formed suspension of nanodroplets in a suitable container (e.g. a vial).

Typically, the operating pressure into the microfluidic cartridge is lower than 300 psi (about 2000 kPa) preferably lower than 100 psi, (about 700 kPa), e.g. between 10 and 90 psi.

An example of microfluidic cartridge is the commercially available NxGen Cartridge, with or without in-line dilution, from Precision Nanosystems (Vancouver, Canada). These microfluidic cartridges can comprise either staggered herringbone or toroidal micromixers, both operating under non-turbulent conditions. For the manufacturing process, the microfluidic cartridge is mounted on a microfluidic instrument, generally equipped with a cartridge adapter, to host the microfluidic cartridge, and with containers (e.g. syringes or vials for continuous-flow injection) directly connected to the inlets of the microfluidic cartridge and specifically designed to pump the liquid phases into said inlets. Example of a microfluidic instrument is the NanoAssemblr® Benchtop Automated Instrument (Precision Nanosystems (Vancouver, Canada)).

An aspect of the invention relates to a method for the preparation of an aqueous suspension of calibrated nanodroplets, said nanodroplets comprising an outer shell and an inner core, wherein said outer shell comprises an amphiphilic component and said inner core comprises a low boiling fluorinated compound, said method comprising:
a) Preparing an aqueous phase;
b) Preparing an organic phase; wherein
   said aqueous phase comprises an amphiphilic component and the organic phase comprises a low boiling fluorinated compound or
   said organic phase comprises an amphiphilic component and a low boiling fluorinated compound;
c) Injecting said aqueous phase into a first inlet and said organic phase into a second inlet of a microfluidic cartridge thereby mixing said aqueous phase and said organic phase in a mixing device of the microfluidic cartridge to obtain an aqueous suspension of calibrated nanodroplets; and
d) Collecting the aqueous suspension of calibrated nanodroplets from the exit channel of the microfluidic cartridge.

According to a preferred embodiment the organic phase comprises an amphiphilic component and a low boiling fluorinated compound.

According to the disclosed method it is possible to obtain an aqueous suspension of calibrated nanodroplets by a single passage of the liquid phases through the microfluidic cartridge mixing portion.

In an embodiment said method for the preparation of an aqueous suspension of calibrated nanodroplets is the microfluidic technique, wherein said calibrated nanodroplets have a Z-average diameter comprised between 100 and 1000 nm and a polydispersity index (PDI) lower than 0.25, preferably lower than 0.20, more preferably lower than 0.15, even more preferably lower than 0.1.

### Step a) Preparing an Aqueous Phase

The expression “aqueous phase” refers to a liquid comprising an aqueous liquid component, including for instance water, aqueous buffered solutions, aqueous isotonic solutions or a mixture thereof. Preferably the aqueous phase is water.

According to an embodiment, said aqueous phase comprises an amphiphilic component as above defined.

For instance, an amphiphilic component can be admixed with an aqueous component through traditional techniques (e.g. stirring) in order to prepare the aqueous phase to be injected into the first inlet of the microfluidic cartridge.

Amphiphilic components suitable to be solubilized in the aqueous phase are polyoxyethylene derivatives, such as polyoxyethylene sorbitan esters (i.e. polysorbates) or polyoxyethylene-polyoxypropylene block copolymers (i.e. poloxamers).

Suitable examples of polyoxyethylene sorbitan esters are polyoxyethylene sorbitan laurate, palmitate, stearate or oleates, such as polysorbate 20 (monolaurate), polysorbate 40 (monopalmitate), polysorbate 60 (monostearate), polysorbate 65 (tristearate) or polysorbate 80 (oleate).

A suitable example of polyoxyethylene-polyoxypropylene block copolymers is Pluronic F68.

In an embodiment, said aqueous phase is a homogenous mixture, i.e. a solution, wherein the amphiphilic component (i.e. the solute) is dissolved in said aqueous liquid component (i.e. the solvent).

Optionally, said aqueous phase comprises a stabilizing component.

The term stabilizing component indicates any compound admixed to the aqueous phase at step a) to further increase the stability of the final calibrated low boiling (P)FC-NDs, without necessarily participating at the formation of the stabilizing layer.

### Step b) Preparing an Organic Phase

The expression “organic phase” refers to an organic solution comprising an organic solvent miscible with water including methanol, ethanol, isopropanol, acetonitrile and acetone. Preferably the organic phase is ethanol.

In the present invention the expression “organic solvent miscible with water” indicates an organic solvent capable of mixing in any ratio (e.g. any concentration) with water without separation of the two phases, i.e. forming a homogeneous solution.

In a preferred embodiment, said organic phase comprises an amphiphilic component. For instance, an amphiphilic component can be admixed with an organic solvent through traditional techniques (e.g. stirring) in order to prepare the organic solution to be mixed with the low boiling fluorinated compound.

In further preferred embodiment, said amphiphilic component is selected from fluorinated surfactants, phospholipids, lipid bearing polymers, fatty acids or a mixture thereof.

Preferably, said amphiphilic component comprises a mixture comprising a phospholipid and a lipid bearing polymers. Still more preferably said mixture further comprises a fatty acid.

In an embodiment, the concentration into the organic phase of said mixture comprising a phospholipid and a lipid bearing polymers is higher than 0.5 mg/mL, preferably at least 2 mg/mL or higher preferably 4 mg/mL or higher, more preferably higher than 5 mg/mL, up to e.g. 10 mg/mL, preferably up to 8 mg/mL.

As disclosed herein, said organic phase comprises a low boiling fluorinated compound. Preferably the low boiling fluorinated compound is a low boiling perfluorocarbon.

Suitable examples of low boiling fluorinated compounds are those mentioned above.

According to the disclosed method, at step b) the preparation of the organic phase to be injected into the second inlet of the microfluidic cartridge can be performed through two alternative approaches.

Said low boiling fluorinated compound may be added to the organic solvent as a liquid (e.g. by dissolution into the organic solvent) or as a gas (e.g. by bubbling into the organic solvent). Preferably said fluorinated compound is a perfluorocarbon.

### Preparing an Organic Phase by Admixing of Low Boiling Fluorinated Compound as a Liquid Phase

According to an embodiment, step b) comprises preparing an organic phase by the addition of a liquid low boiling fluorinated compound into an organic solvent.

In an embodiment, said step b) relates to a method for the preparation of an organic phase comprising a low boiling fluorinated compound, said method comprising the steps of:
b.1) Condensing a gaseous low boiling fluorinated compound to obtain a liquid low boiling fluorinated compound;
b.2) Preparing a liquid organic solution comprising an organic solvent;
b.3) Cooling said liquid organic solution to a temperature below the boiling point of said liquid low boiling fluorinated compound;
b.4) Mixing said liquid low boiling fluorinated compound and said liquid organic solution to obtain an organic phase comprising a liquid low boiling fluorinated compound.

In a preferred embodiment, step b.1) is performed by cooling said gaseous low boiling fluorinated compound down to a temperature below the boiling point of said low boiling fluorinated compound.

In a further embodiment, at step b.4) the temperature at which the mixing is performed is a temperature below the boiling point of said low boiling fluorinated compound.

Preferably, the temperature at step b.4) is the same temperature of step b.3).

Still more preferably the temperature at step b.1), step b.3) and step b.4) is suitable to avoid or substantially limit the evaporation of said liquid low boiling fluorinated compound until to the injection of said organic phase into the microfluidic cartridge (i.e. step c).

The temperature at step b.1), step b.3) and step b.4) is lower than the boiling point of said low boiling fluorinated compound, preferably it is at least 5° C. lower than the boiling point of said low boiling fluorinated compound, more preferably at least 10° C. lower, even more preferably at least 20° C. lower, up to e.g. 50° C. lower.

The temperature of the obtained organic phase at the end of step b.4) is thus lower than the boiling point of said low boiling fluorinated compound, preferably it is at least 5° C. lower than the boiling point of said low boiling fluorinated compound, more preferably at least 10° C. lower, even more preferably at least 20° C. lower, up to e.g. 50° C. lower.

At step b.4) said mixing is performed for a time suitable to allow the dissolution of said liquid low boiling fluorinated compound into said liquid organic solution, e.g. within 5 minutes. At the end of said mixing, an organic phase is obtained in the form of a homogenous solution. Said organic phase shall be injected into the microfluidic cartridge within a time suitable for avoiding or substantially limiting the evaporation of the low boiling fluorinated compound from the organic phase, e.g. within 5 minutes from the end of step b.

In a further embodiment, at step b.4) the organic phase comprises a low boiling fluorinated compound at a concentration ranging between 5 and 100 μL/mL, preferably between 10 and 50 μL/mL, still more preferably between 15 and 30 μL/mL.

### Preparing an Organic Phase by Admixing the Low Boiling Fluorinated Compound as a Gaseous Phase

According to an alternative embodiment, step b) comprises preparing an organic phase by the addition of a gaseous low boiling fluorinated compound into an organic solvent.

In an embodiment, said step b) relates to a method for the preparation of an organic phase comprising a low boiling fluorinated compound, said method comprising the steps of:
b.i) Preparing a liquid organic solution comprising an organic solvent, and
b.ii) Admixing a gaseous low boiling fluorinated compound to said organic liquid solution.

Preferably at step b.ii) the admixing is performed by bubbling the gaseous low boiling fluorinated compound into the organic solution.

According to a preferred embodiment, before the admixing of step b.ii) the temperature of the organic solution is set above the boiling point of said gaseous low boiling fluorinated compound.

Preferably, said temperature is between the boiling point and 25° C., more preferably up to 15° C. higher than the boiling point, even more preferably up to 10° C. higher.

The temperature of the obtained organic phase at the end of step b.ii) is above the boiling point of said low boiling fluorinated compound.

Preferably, said temperature is between the boiling point and RT, more preferably up to 15° C. higher than the boiling point, even more preferably up to 10° C. higher.

At step b.ii) the concentration of the low boiling fluorinated compound into the organic phase can be any concentration, preferably up to the saturation concentration. Preferably the concentration of said low boiling fluorinated compound is the saturation concentration.

The saturation concentration of a fluorinated compound depends on the solvent used and on the temperature. For instance, considering ethanol as solvent, the saturation concentration of perfluorobutane is 2.5% by volume and the saturation concentration of perfluropropane is 2.7% by volume (US2019307908A1).

Preferably, at step b.ii) said admixing is performed for a time suitable to reach the saturation concentration of the gaseous low boiling fluorinated compound into the organic liquid solution, e.g. for 2 minutes.

At the end of the admixing, an organic phase consisting of a homogenous solution is obtained.

In a further embodiment, at the end of its preparation process, said organic phase can be diluted by adding a suitable amount of organic solvent in order to reduce the (P)FC concentration into the organic phase.

The expression “suitable amount of organic solvent” indicates the quantity of organic solvent (e.g. in mL) necessary to reduce the initial concentration of (P)FC into the organic phase (e.g. the saturation concentration).

Said organic phase is then injected into the microfluidic cartridge.

Step c) Injection into the Microfluidic Cartridge

Typically, at step c) the injection of the aqueous phase and the injection of the organic phase are carried out simultaneously.

The expression “simultaneously” indicates the simultaneous injection (i.e. co-injection) of the aqueous phase and the organic phase into the microfluidic cartridge, that is to say that the aqueous phase and organic phase are injected into two separate inlets of the microfluidic cartridge at the same time or at substantially the same time (e.g. within few seconds).

In a preferred embodiment, both aqueous and organic phases are injected into the microfluidic cartridge at a temperature suitable to avoid or substantially limit the evaporation of the low boiling fluorinated compound. For instance, after their respective preparations (i.e. step a) and step b)) both the aqueous phase and the organic phase can be stored in an ice bath (about 4° C.) before their injection into the separate inlets of the microfluidic cartridge (preferably said injection occurs not later than 3 minutes from the preparation of the two phases), in order to limit the temperature increase during the time between step a), step b) and the subsequent step c).

According to the present invention, after their injections, the aqueous phase and the organic phase are directed towards a mixing device (103 in FIG. 1), wherein they are mixed (e.g. through laminar mixing in the case of a staggered herringbone micromixer (see FIG. 2)) endowing to the formation of NDs.

The temperature of the mixing portion 103, wherein the mixing process takes place into the peculiar micro-channel geometry of the mixing portion, is typically higher than 0° C., preferably it can be comprised between 0° C. and 25° C., preferably comprised between 0° C. and 15° C., more preferably between 0° C. and 5° C.

For instance, the microfluidic cartridge can be stored in the fridge (e.g. 4° C.), for a suitable time able to reach the desired temperature.

### Step d) Collection of the Aqueous Suspension of Calibrated Low Boiling Fluorinated Compound

According to the step d) of the disclosed method, at the end of the mixing process, an aqueous suspension of calibrated nanodroplets is collected from the exit channel of the microfluidic cartridge.

After their collection, the inner core of said microfluidically-obtained low boiling (P)FC-NDs is composed by a low boiling fluorinated compound in a liquid state independently from the temperature at which they are collected and subsequently stored.

In other words, even if the collection step is performed (and the subsequent storage is effected) at a temperature above the boiling point of the (P)FC used for the manufacturing of the calibrated NDs, said (P)FC remains in a liquid state due to its incorporation into the outer stabilizing shell comprising an amphiphilic component. Nevertheless, it is preferable that such temperature is not excessively high, in order to limit possible partial phase transition of the entrapped (P)FC.

According to an embodiment, the temperature at which said collection step is performed can be comprised between 0° C. and 25° C., preferably comprised between 0° C. and 15° C., more preferably between 0° C. and 5° C.

Preferably, the collected aqueous suspension of calibrated low boiling (P)FC-NDs has a temperature comprised between 0° C. and 25° C., preferably comprised between 0° C. and 15° C., more preferably between 0° C. and 5° C.

For instance, after the collection said aqueous suspension can be stored at 4° C. in a fridge.

### Total Flow Rate (TFR) and Flow Rate Ratio (FRR)

The method of the present invention allows to control the low boiling fluorinated compound nanodroplets characteristics by varying two process parameters: the Total Flow Rate and the Flow Rate Ratio.

The expression “Total Flow Rate (TFR)” refers to the total flow of both fluid streams, namely the aqueous phase and the organic phase, being pumped through the two separate inlets of the microfluidic cartridge. The unit of measurement of the TFR is mL/min.

According to an embodiment, the TFR is preferably comprised between 2 mL/min and 18 mL/min, more preferably between 5 mL/min and 16 mL/min, still more preferably the TFR is 10 mL/min.

The expression “Flow Rate Ratio (FRR)” refers to the ratio between the amount of aqueous phase and the amount of organic phase flowing into the microfluidic cartridge, according to the Equation 1:

$$Flow rate ratio = \frac{volume of aqueous phase}{volume of organic phase} \qquad Eq \text{.1}$$

The volume of aqueous and organic phases can be expressed as e.g. mL.

In a preferred embodiment, the FRR (volume of aqueous phase vs. volume of organic phase) is between 1:1 to 5:1, preferably between 1:1 and 3:1, more preferably the FRR is 1:1.

### Optional Step e) Dilution

According to present invention, the method of preparation further comprises an optional step e), which comprises diluting the collected aqueous suspension of calibrated nanodroplets.

As observed by the Applicant, the dilution step after the NDs production, using a microfluidic cartridge, may have a favourable effect on the initial NDs size and initial monodispersity.

As indicated above, the expressions “initial monodispersed distribution” and “initial NDs sizes” refer to the values of monodispersity and NDs sizes of the calibrated NDs composition at the end of its preparation process, wherein said end of the preparation process refers either to i) the collection of the calibrated NDs from the exit channel of the microfluidic cartridge or ii) the collection of said calibrated NDs followed by a dilution step (i.e. step e).

In the present description and claims the term “dilution” refers to the process of reducing the concentration of calibrated nanodroplets in the suspension, by adding a suitable amount of water or of an aqueous solution.

Suitable examples of aqueous solutions are saline (which may advantageously be balanced so that the final product for injection is not hypotonic), or solutions of one or more tonicity adjusting substances.

A suitable amount of water or of an aqueous solution corresponds to the quantity of aqueous solution necessary to reduce the concentration of the calibrated nanodroplets in the aqueous suspension from 2 to 10-folds.

In a preferred embodiment, the optional step e) of the present method comprises diluting the collected aqueous suspension of calibrated nanodroplets from 1 to 20-folds, preferably from 3 to 8-folds, still more preferably the collected aqueous suspension is diluted 5-fold.

An additional effect of dilution is that of reducing the relative amount of organic solvent in the suspension.

Preferably, the optional step e) is performed at a temperature suitable to obtain a diluted aqueous suspension of calibrated low boiling (P)FC-NDs having a temperature comprised between 0° C. and 25° C., preferably comprised between 0° C. and 15° C., more preferably between 0° C. and 5° C.

The diluting step can be alternatively performed inside the microfluidic cartridge, by means of an additional channel (e.g. placed between the mixing device 103 and the exit channel 104 in FIG. 1) aimed at diluting the calibrated NDs suspension before their direction to the exit channel.

In this case, the step e) of the present method comprises diluting the suspension of calibrated nanodroplets from 1 to 20-folds, preferably from 3 to 8-folds, still more preferably the collected aqueous suspension is diluted 5-fold, before their collection from the microfluidic cartridge.

### Stability

In the present description and claims, the term “stability” indicates the property of a nanodroplets composition to substantially maintain over time its initial NDs sizes and preferably also its initial monodispersed distribution.

Initial NDs sizes and initial monodispersed distribution refer to the values of NDs sizes and monodispersity of the calibrated low boiling PFC-NDs composition at the end of the preparation process.

For the sake of clarity, end of the preparation process refers either to i) the collection of the calibrated NDs from the exit channel of the microfluidic cartridge or ii) the collection of said calibrated NDs followed by a dilution step (i.e. step e).

Both these alternative final stages are performed before any storage period of the calibrated NDs.

The expression “storage period” indicates a period of time, e.g. expressed as hours, days or weeks, during which a microfluidically-prepared aqueous suspension of calibrated low boiling (P)FC-NDs is kept under certain conditions of temperature after the end of the preparation process.

It is possible to calculate the stability of the calibrated nanodroplets by using the NDs size evolution (% Evol) parameter, according to the following equation (Eq.2):

$$\% Evol = \frac{( Dfinal - Dinitial )}{Dinitial} \times 100 \qquad Eq \text{.2}$$

### Wherein:

D final is the z-average diameter of the calibrated NDs after a certain time from the end of the preparation process, e.g. after 60 minutes from the end of preparation process or after a storage period (e.g. one week) at different conditions (different temperatures, pressure, etc); and
D initial is the z-average diameter of the calibrated NDs immediately (e.g. within minutes) at the end of its preparation process.

In the present invention, a value of % Evol close to 0 (either positive or negative) indicates a higher stability of the calibrated NDs suspension, whereby the nanodroplets in the suspension substantially maintain over time their initial mean dimensions.

According to the present invention, the % Evol of a calibrated NDs suspension is preferably lower than ±50%, more preferably lower than ±30%, and still more preferably is lower than ±20%.

### Aqueous Suspension of a Calibrated (P)FC-NDs

An aspect of the invention relates to an aqueous suspension comprising a plurality of calibrated nanodroplets, said nanodroplets comprising an outer shell and an inner core, wherein said outer shell comprises an amphiphilic component and said inner core comprises a low boiling fluorinated compound, such suspension obtained according to the process as above defined.

A preferred embodiment relates to an aqueous suspension comprising a plurality of calibrated nanodroplets, as above defined, wherein said nanodroplets have a z-average diameter comprised between 100 nm and 1000 nm and a polydispersity index lower than 0.25.

In the present description and claims, the term “plurality of calibrated low boiling (P)FC nanodroplets” refers to a population of nanodroplets characterized by a calibrated distribution, meaning that substantially all the vesicles have substantially similar sizes.

The expression “calibrated distribution” indicates a certain population of nanodroplets (e.g. with a z-average diameter comprised between 100 and 1000 nm) with a polydispersity index (PDI) lower than 0.25, preferably lower than 0.2, more preferably lower than 0.15, even more preferably lower than 0.1.

The term “polydispersity” (PDI) refers to a dimensionless measure of the broadness of the size distribution calculated from the cumulants analysis, wherein said cumulants analysis, defined in the International Standard on Dynamic Light Scattering ISO13321 (1996) and ISO22412 (2008), gives a mean particle size (z-average) and an estimate of the width of the distribution (polydispersity index).

For instance, a polydispersity higher than 0.7 indicates a very broad distribution of particles sizes, while a value lower than 0.08 indicates a nearly “perfect” monodisperse sample characterized by a substantial monomodal distribution of the particles sizes. The polydispersity can be measured with the dynamic light scattering technique (DLS), by using for instance the Malvern Zetasizer Nano-ZS instrument (Malvern Instruments Ltd., UK).

The “z-Average Diameter (ZD)” is defined as the intensity-weighted mean diameter derived from the cumulants analysis. In other words, it relates to the average of calibrated vesicles size dispersed in the aqueous suspension measured through the dynamic light scattering technique (DLS).

In the present invention, the z-average diameter is comprised between 100 nm and 1000 nm, preferably between 120 and 600, more preferably between 150 and 400.

The aqueous suspension comprising a plurality of calibrated nanodroplets further comprises an aqueous component as above defined. Suitable aqueous components for said aqueous suspension are preferably physiologically acceptable comprising water (preferably sterile water), saline (which may advantageously be balanced so that the final product for injection is not hypotonic), aqueous buffered solution or aqueous isotonic solution.

### Use

The acoustic droplet vaporization (ADV) is a phenomenon through which (P)FC-NDs can be converted into gas microbubbles upon exposure to ultrasound energy beyond the vaporization threshold.

When administered invivo, PCCA nanodroplets present many advantages with respect to traditional microbubbles, such as inertness, relatively low toxicity, relative stability in circulation, immiscibility in water, and low surface tension. Once vaporized, the generated microbubbles can be effectively used in either imaging or therapeutic applications with ultrasound, including sonopermeabilization, thermal ablation, blood brain barrier (BBB) disruption, multimodal imaging modalities and allow passive (due to the enhanced permeability and retention (EPR) effect in the tumor tissues) or active targeting (by incorporating targeted ligands) for localized delivery of therapeutic drugs or genes. Another potentially valuable characteristic of PFC-NDs is their possible application for novel imaging strategies such as UltraSound Super-Resolution Imaging since these agents can be activated and deactivated on demand by applying intermittent acoustic pulses.

Considering the low boiling (P)FC-NDs of the present invention, the presence of a low boiling fluorinated compound in the inner core makes the resulting droplets acoustically activatable with substantially less energy than other phase-change contrast agents, e.g. similarly-sized nanodroplets comprising higher boiling point fluorinated compound, endowing to considerable advantages for their applications in diagnostics, therapeutics and other treatments.

A further aspect relates to an aqueous suspension comprising a plurality of calibrated low boiling (P)FC-NDs obtained according to the process as above defined for use in a diagnostic and/or therapeutic treatment.

Diagnostic treatment includes any method where the use of the nanodroplets allows enhancing the visualisation of a portion or of a part of an animal (including humans) body, including imaging for preclinical and clinical research. Suitable examples of diagnostic applications are molecular and perfusion imaging, tumor imaging (EPR effect), multimodal imaging (MR-guided tumor ablation, fluorescence, sono-photoacoustic activation), US aberration correction and super-resolution imaging.

Therapeutic treatment includes any method of treatment of a patient. In preferred embodiments, the treatment comprises the combined use of ultrasounds and (P)FC-NDs either as such (e.g. in ultrasound-mediated thrombolysis, high intensity focused ultrasound ablation, blood-brain barrier permeabilization, immunomodulation, neuromodulation, radiosensitization) or in combination with a therapeutic agent (i.e. ultrasound-mediated delivery, e.g. for the delivery of a drug or bioactive compound to a selected site or tissue, such as in tumor treatment, gene therapy, infectious diseases therapy, metabolic diseases therapy, chronic diseases therapy, degenerative diseases therapy, inflammatory diseases therapy, immunologic or autoimmune diseases therapy or in the use as vaccine), whereby the presence of the vesicles may provide a therapeutic effect itself or is capable of enhancing the therapeutic effects of the applied ultrasounds, e.g. by exerting or being responsible to exert a biological effect in vitro and/or in vivo, either by itself or upon specific activation by various physical methods (including e.g. ultrasound-mediated delivery).

The following examples will help to further illustrate the invention.

### EXAMPLES

### Materials and Methods

The following materials are employed in the subsequent examples:

| | | | Formula |
|---|---|---|---|
| | | | weight |
| Material | | Molecular formula | (g/mol) |
| Zonyl | Nonionic fluorosurfactant | (C₂H₄O)ₓ(CF₂)yC₂H₅FO | 725 |
| FSO | | | |
| DPPC | 1,2-dipalmitoyl-sn- | C₄₀H₈₀NO₈P | 734 |
| | glycero-3-phosphocho- | | |
| | line | | |
| DPPE- | 1,2-dipalmitoyl-sn- | C₂₆₅H₅₃₁N₂O₁₂₃P | 5745 |
| PEG5000 | glycero-3-phosphoeth- | | |
| | anolamine-N-[me- | | |
| | thoxy(polyethylene | | |
| | glycol)-5000] | | |
| | (ammonium salt) | | |
| DMPA | 1,2-dimyristoyl-sn- | C₃₁H₆₀O₈PNa | 614 |
| | glycero-3-phosphate | | |
| | (sodium salt) | | |
| PA | Palmitic acid | C₁₆H₃₂O₂ | 256 |
| C3F8 | perfluoropropane | C₃F₈ | 188 |
| C4F10 | perfluorobutane | C₄F₁₀ | 238 |
| Ethanol | Ethyl alcohol | C₂H₅OH | 46 |

### Example 1

### Preparation of the Organic Phase by Admixing the Low Boiling Fluorinated Compound as a Liquid Phase

### 1.1 Organic Phase Comprising Perfluorobutane

A solution of Zonyl FSO at 2 mg/mL in ethanol was prepared and it was cooled down to a temperature of −20° C. Then, liquid perfluorobutane (C₄F₁₀, b.p. −2° C.) was obtained by condensation of PFB gas into a syringe at −20° C. and was added to the Zonyl FSO ethanolic solution at a concentration of 25 μL/mL. The solution was stirred until the total dissolution of liquid perfluorobutane.

### 1.2 Organic Phase Comprising Perfluoropropane

An organic solution was prepared by adding a lipid mixture of DPPC/PA/DPPE-PEG5k (molar ratio of 74.1/18.5/7.4) at 6 mg/mL in ethanol and it was cooled down to a temperature of −80° C. Then, liquid perfluoropropane (C₃F₈, b.p. −37° C.) was obtained by condensation of perfluoropropane gas into a syringe at −80° C. and was added to the lipid ethanolic solution at a concentration of 25 μL/mL. The solution was stirred until the total dissolution of liquid perfluoropropane.

### Example 2

### Preparation of the Organic Phase by Admixinq the Low Boiling Fluorinated Compound as a Gaseous Phase

### 2.1 Organic Phase Comprising Perfluorobutane

An organic solution was prepared by adding a lipid mixture of DPPC/PA/DPPE-PEG5k (molar ratio of 74.1/18.5/7.4) at 6 mg/mL in ethanol. Subsequently, the temperature of the ethanolic solution was cooled down in an ice bath. Finally, the perfluorobutane gas (C₄F₁₀) was introduced into said ethanolic solution, kept in the ice bath, by bubbling until gas saturation (˜25 μL/mL).

### 2.2 Organic Phase Comprising Perfluoropropane

An organic solution was prepared by adding a lipid mixture of DPPC/PA/DPPE-PEG5k (molar ratio of 74.1/18.5/7.4) at 6 mg/mL in ethanol. Subsequently, the temperature of the ethanolic solution was cooled down to −20° C. Finally, the perfluoropropane gas (C₃F₈) was introduced into said ethanolic solution by bubbling until gas saturation (˜27 μL/mL), at −20° C.

### Example 3

### General Procedure for the Preparation of Low Boiling Point PFC Nanodroplets Using a Microfluidic Platform

Low boiling point PFC nanodroplets were formulated with a NanoAssemblr™ Benchtop automated instrument from Precision Nanosystems (Vancouver, Canada) equipped with a staggered herringbone micromixer (SHM) allowing size-controlled self-assemblies. Briefly, an aqueous phase was injected into the first inlet whereas the organic phase, prepared according to Example 1 or Example 2, comprising a low boiling point PFC dissolved in the organic solvent, was injected into the second inlet of the microfluidic cartridge (FIG. 1). Both phases were placed into an ice bath at about 4° C. before the NDs formulation. Microscopic characteristics of the channels are engineered to cause an accelerated mixing of the two fluid streams in a controlled fashion. The microfluidic process settings, namely the Total Flow Rate (TFR, in mL/min) and the Flow Rate Ratio (FRR), were varied to control the NDs characteristics. The NDs suspensions were collected from the exit channel in a Falcon vial (15 mL). The collected aqueous suspension of calibrated low boiling point PFC nanodroplets was then diluted with ultrapure water, previously placed in an ice bath.

The specific type and amounts of materials used in the preparations illustrated in the following examples are summarized in Table 1.

TABLE 1
Compositions used in the present Examples

| | | | Organic phase | | | |
|---|---|---|---|---|---|---|
| | Aqueous phase | | | | Low boiling | Method of |
| | | Amphiphilic | | Amphiphilic | fluorinated | preparation of |
| | | material | | material | compound | the organic |
| Compositionnr | Solvent | (type; conc) | Solvent | (type; conc) | (type; conc) | phase |
| 1 | MilliQ | — | Ethanol | Zonyl FSO; | Perfluorobutane; | Condensation |
| | water | | | 2 mg/mL | 25 μL/mL | |
| 2 | MilliQ | — | Ethanol | DPPC/PA/DPPE- | Perfluorobutane; | Condensation |
| | water | | | PEG5k (molar | 25 μL/mL | |
| | | | | ratio of | | |
| | | | | 74.1/18.5/7.4); | | |
| | | | | 6 mg/mL | | |
| 2* | MilliQ | — | Ethanol | DPPC/PA/DPPE- | Perfluorobutane; | Bubbling |
| | water | | | PEG5k (molar | saturation | |
| | | | | ratio of | | |
| | | | | 74.1/18.5/7.4); | | |
| | | | | 6 mg/mL | | |
| 3 | MilliQ | — | Ethanol | DPPC/DMPA/DPPE- | Perfluorobutane; | Condensation |
| | water | | | PEG5000 (molar | 25 μL/mL | |
| | | | | ratio of | | |
| | | | | 82.5/10/7.5); | | |
| | | | | 6 mg/mL | | |
| 4 | MilliQ | — | Ethanol | DPPC/PA/DPPE- | Perfluoropropane; | Condensation |
| | water | | | PEG5k (molar | 25 μL/mL | |
| | | | | ratio of | | |
| | | | | 74.1/18.5/7.4); | | |
| | | | | 6 mg/mL | | |
| 4* | MilliQ | — | Ethanol | DPPC/PA/DPPE- | Perfluoropropane; | Bubbling |
| | water | | | PEG5k (molar | saturation | |
| | | | | ratio of | | |
| | | | | 74.1/18.5/7.4); | | |
| | | | | 6 mg/mL | | |

### Example 4

Effect of the method of preparation of the organic phase Aqueous suspensions of calibrated low boiling PFC nanodroplets were prepared through the microfluidic method as described in Example 3, the set processing parameters being FRR 1-1 and TFR 10 mL/min.

In order to investigate the influence of the different methods of preparation of the organic phase on the size, the PDI and the stability of the final PFC NDs, compositions obtained by preparing the organic phase through both condensation and bubbling method were tested. In particular Composition 2 (perfluorobutane/condensation) was compared with Composition 2* (perfluorobutane/bubbling) and Composition 4 (perfluoropropane/condensation) was compared with Composition 4* (perfluoropropane/bubbling).

After their collection from the microfluidic cartridge, the aqueous suspensions of low boiling PFC nanodroplets were directly diluted with water (4-folds) and consequently characterized using a Malvern Zetasizer Nano-ZS instrument (Malvern Instruments Ltd., UK) to measure the size and size distribution (PDI). The characterization was performed immediately after the dilution step (e.g. within 5 minutes) and after a one-week storage at 4° C.

### Results

TABLE 2
Effect of the method of preparation of the organic
phase on the sizes, PDI and % Evol (size) of
a composition comprising perfluorobutane

| | Initial sizes | | |
|---|---|---|---|
| | (nm) | PDI | % Evol |
| Composition 2 | 208 | 0.177 | 7% |
| Composition 2* | 202 | 0.165 | 5% |

TABLE 3
Effect of the method of preparation of the organic
phase on the sizes, PDI and % Evol (size) of
a composition comprising perfluoropropane

| | Initial sizes | | |
|---|---|---|---|
| | (nm) | PDI | % Evol |
| Composition 4 | 246 | 0.144 | −3% |
| Composition 4* | 231 | 0.173 | 4% |

The overall results of the study of the influence of the preparation method of the organic phase on the calibrated low boiling PFC nanodroplets are displayed in Table 2 and Table 3, which show the characterization results obtained for 4 different Compositions (i.e. Composition 2, Composition 2*, Composition 4 and Composition 4*). The characterization was performed immediately after the dilution of the aqueous suspensions and after one-week storage at 4° C.

In particular, the variation of the NDs sizes over time is expressed as % Evol, calculated following Equation 2, as described in the Description section.

A value of % Evol close to 0 indicates a higher stability of the calibrated NDs suspension whereby the nanodroplets in the suspension substantially maintain over time its initial mean dimensions.

The results displayed that good PDI values combined with an improved NDs stability (i.e. low % Evol) over time were obtained independently from the method used to prepare the organic phase of the disclosed method.

### Example 5

### Effect of the Flow Rate Ratio

Aqueous suspensions of calibrated low boiling PFC nanodroplets were prepared according to the microfluidic method as described in Example 3, by modifying the respective flow rate ratios.

In order to investigate the effect of the Flow Rate Ratio (FRR) on the size, the PDI and the stability of the PFC NDs, three different FRR were tested for the preparation of the Composition 1, Composition 2 and Composition 3. In particular, the total flow rate (TFR) was set to 10 mL/min and the Flow rate ratio (FRR) was varied from 1-1 to 3-1 (aqueous/organic).

After their collection from the microfluidic cartridge, the aqueous suspensions of low boiling PFC nanodroplets were directly diluted with water (4-folds) and consequently characterized using a Malvern Zetasizer Nano-ZS instrument (Malvern Instruments Ltd., UK) to measure the size and size distribution (PDI). The characterization was performed immediately after the dilution step (e.g. within 5 minutes) and after a one-week storage at 4° C.

### Results

TABLE 4
Effect of FRR on the sizes, PDI and % Evol (size) of Composition 1
Composition 1

| | Initial sizes | | |
|---|---|---|---|
| FRR | (nm) | PDI | % Evol |
| 1-1 | 334 | 0.040 | −6% |
| 2-1 | 215 | 0.035 | 247% |
| 3-1 | 218 | 0.035 | 239% |

TABLE 5
Effect of FRR on the sizes, PDI and % Evol (size) of Composition 2
Composition 2

| | Initial sizes | | |
|---|---|---|---|
| FRR | (nm) | PDI | % Evol |
| 1-1 | 212 | 0.171 | 12% |
| 2-1 | 272 | 0.430 | −47% |
| 3-1 | 354 | 0.305 | 33% |

TABLE 6
Effect of FRR on the sizes, PDI and % Evol (size) of Composition 3
Composition 3

| | Initial sizes | | |
|---|---|---|---|
| FRR | (nm) | PDI | % Evol |
| 1-1 | 266 | 0.130 | −5% |
| 2-1 | 286 | 0.507 | 283% |
| 3-1 | 554 | 0.154 | 324% |

The overall results of the study of the effect of the FRR on the calibrated low boiling PFC nanodroplets are displayed in Table 4, Table 5 and Table 6, which show the characterization results obtained for three different Compositions (i.e. Composition 1, Composition 2 and Composition 3). The second column of the Tables displays the FRR ratio at which the compositions were prepared. The characterization was performed immediately after the dilution of the aqueous suspensions from the microfluidic cartridge and after one-week storage at 4° C.

In particular, the variation of the NDs sizes over time is expressed as % Evol, calculated following Equation 2, as described in the Description section.

A value of % Evol close to 0 indicates a higher stability of the calibrated NDs suspension whereby the nanodroplets in the suspension substantially maintain over time its initial mean dimensions.

The results displayed that using a FRR of 1-1 (aqueous phase-organic phase) endowed to good PDI values combined with an improved NDs stability (i.e. low % Evol) over time in all the three investigated Compositions.

Increasing the amount of aqueous phase, in comparison with the organic phase, mainly resulted in a reduction of the NDs stability over time.

In addition to this, Composition 2, comprising a mixture of phospholipids, a pegylated phospholipid and a fatty acid, resulted to be substantially stable at any investigated FRR, with a % Evol lower than ±50%.

### Example 6

### Repeatability of the Microfluidic Method

In order to assess the repeatability of the disclosed microfluidic method, multiple formulations of calibrated low boiling point PFC nanodroplets were prepared as described in Example 3 and subsequently characterized.

The term repeatability indicates a measure of the ability of the microfluidic method to generate similar results for multiple preparations of the same sample.

For this purpose, Composition 2 (perfluorobutane—organic phase prepared by condensation method, Example 1) and Composition 4* (perfluoropropane—organic phase prepared by bubbling method, Example 2) were prepared four times each, the set processing parameters being FRR 1-1 and TFR 10 mL/min.

Immediately after their collection from the microfluidic cartridge outlet, the samples were diluted 4-folds using water and were characterized using a Malvern Zetasizer Nano-ZS instruments in order to determine the NDs sizes (Z-Average) and polydispersity (PDI). The characterization was performed immediately after the dilution step (e.g. within 5 minutes) and after a one-week storage at 4° C.

### Results

TABLE 7
Characterization of microfluidically-prepared
multiple formulations of Composition 2
Composition 2

| | Initial size | | |
|---|---|---|---|
| FRR | (nm) | PDI | % Evol |
| 1-1 | 212 | 0.171 | 12% |
| | 222 | 0.171 | 7% |
| | 208 | 0.177 | 7% |
| | 271 | 0.167 | −14% |

TABLE 8
Characterization of microfluidically-prepared
multiple formulations of Composition 4*
Composition 4*

| | Initial size | | |
|---|---|---|---|
| FRR | (nm) | PDI | % Evol |
| 1-1 | 231 | 0.173 | 4% |
| | 265 | 0.186 | 1% |
| | 247 | 0.140 | 0% |
| | 239 | 0.141 | 1% |

The characterization of the microfluidically-prepared multiple formulations having the same composition (Table 7 and Table 8) demonstrated that the repeated samples including low boiling PFC-NDs displayed close characteristics in terms of size and PDI and a similar stability behavior over time, independently from the method adopted for the preparation of the organic phase. In particular, the variation of the NDs sizes over time is expressed as % Evol, calculated following Equation 2, as described in the Description section.

These results confirmed the good repeatability of the disclosed microfluidic method.

### Example 7

### Influence of Palmitic Acid Amount in the Lipid Mixture

In order to evaluate the influence of the palmitic acid amount on the size and PDI of the calibrated low boiling point PFC nanodroplets, different aqueous suspensions of calibrated nanodroplets were prepared through the microfluidic method as described in Example 3, the set processing parameters being FRR 1-1 and TFR 10 mL/min, testing different percentages of palmitic acid in the lipid mixture.

For this purpose, Composition 2 was selected, and the molar ratio of the palmitic acid was varied from 0% to 70% while keeping the DPPE-PEG5000 percentage constant, to obtain the following Compositions:

TABLE 9
Compositions having increasing amount of palmitic acid (PA)

| | Molar ratio | | |
|---|---|---|---|
| Composition nr | DPPC | PA | DPPE-PEG5k |
| 2a | 92.6 | 0 | 7.4 |
| 2 | 74.1 | 18.5 | 7.4 |
| 2b | 46.3 | 46.3 | 7.4 |
| 2c | 22.6 | 70 | 7.4 |

After their collection from the microfluidic cartridge, the aqueous suspensions of low boiling point PFC nanodroplets were directly diluted with water (4-folds) and consequently characterized using a Malvern Zetasizer Nano-ZS instrument (Malvern Instruments Ltd., UK) to measure the size and size distribution (PDI). The characterization was performed immediately after the dilution step (e.g. within 5 minutes) and after a one-week storage at 4° C.

### Results

TABLE 10
Influence of the palmitic acid amount
on the size, PDI and % Evol (size)

| Composition | Palmitic acid | Initial size | | |
|---|---|---|---|---|
| nr | (molar ratio %) | (nm) | PDI | % Evol |
| 2a | 0 | 500 | 0.106 | −13% |
| 2 | 18.5 | 212 | 0.171 | 12% |
| 2b | 46.3 | 291 | 0.089 | 1% |
| 2c | 70 | 332 | 0.318 | 31% |

Table 10 shows the size and PDI characterization of four different compositions of low boiling PFC-NDs comprising an increasing amount of palmitic acid. Since the characterization was performed immediately after the dilution step and after a one-week storage at 4° C., the variation of the NDs sizes over time is expressed as % Evol, calculated following Equation 2, as described in the Description section.

Results demonstrated that the presence of palmitic acid in the formulation played a role in controlling sizes and PDI of the respective compositions. In particular, Composition 2a, not comprising palmitic acid, displayed higher NDs size in comparison with the other formulations having increasing amount of palmitic acid.

Furthermore, a high molar ratio of palmitic acid, i.e. Composition 2c, negatively affected both PDI and stability of the aqueous suspension of calibrated NDs.

Composition 2 and Composition 2b, respectively comprising 18.5 and 46.3% of molar ratio in the lipid mixture, endowed to good values of both size and PDI, further displaying a good stability over time.

### Example 8

### Determination of Acoustic Droplet Vaporization (ADV)

The expression “Acoustic Droplet Vaporization (ADV) threshold” indicates the minimal acoustic pressure that is necessary to obtain the nanodroplets conversion into echogenic microbubbles.

The Acoustic Droplet Vaporization (ADV) threshold of the NDs prepared according to the previous examples can be determined according to conventional methodologies using B-mode imaging methods. For instance, the suspension of NDs can be vaporized while passing through the focal zone of a transducer and the acoustic pressure is increased of about 0.2 MPa each 5 s until the NDs vaporization is observed.

Nanodroplets activation was performed by focused ultrasound waves on five aligned focal points allowing the activation only within the region of interest where the acoustics pressure was highest. Pulses were emitted in burst mode at a frequency of 6 MHz, 20 cycles per pulse and at a pulse-repetition frequency (PRF) of 1 Hz.

The acoustic pressure was increased every 5 s until the observation of the NDs vaporization.

For the ADV determination, two different compositions were tested (see Table 9). In particular, Composition 2 (Perfluorobutane; condensation method) was compared with a microfluidically-obtained PFC-NDs composition having the same shell composition (i.e. DPPC/PA/DPPE-PEG5k at 74.1/18.5/7.4) comprising a perfluorocarbon with higher boiling points (i.e. liquid at room temperature), i.e. perfluoropentane (boiling point 29° C.).

### Results

Table 11 reports the overall results obtained from the determination of the ADV thresholds. Each value is the average of three successive determinations at a NDs concentration of 1.7×10⁺⁸ P/mL.

TABLE 11
Acoustic Droplet Vaporization (ADV) threshold
as a function of the PFC core

| Composition | | | | |
|---|---|---|---|---|
| nr | Amphiphilic component | PFC | MPa | SD |
| 2 | DPPC/PA/DPPE-PEG5k | C₄F₁₀ | 5.08 | 0.187 |
| 5 | (molar ratio 74.1/18.5/7.4) | C₅F₁₂ | 8.25 | 0.126 |

From the comparison, it emerges that for NDs comprising a PFC having a high boiling point a higher ADV threshold was obtained than the composition comprising the low boiling PFC.

### REFERENCES

Durham P. G., Dayton P. A, Current Opinion in Colloid & Interface, 56, 2021, 101498
Sheeran P. S. et al, Langmuir, 27, 2011, 10412-10420
US 2019/0307908
Melich R. et al, International Journal of Pharmaceutics, 587, 2020, 119651

1. A method for the preparation of an aqueous suspension of calibrated nanodroplets, said nanodroplets comprising an outer shell and an inner core, wherein said outer shell comprises an amphiphilic component and said inner core comprises a low boiling fluorinated compound, said method comprising:
a) Preparing an aqueous phase;
b) Preparing an organic phase; wherein
said aqueous phase comprises an amphiphilic component and the organic phase comprises a low boiling fluorinated compound or
said organic phase comprises an amphiphilic component and a low boiling fluorinated compound;
c) Injecting said aqueous phase into a first inlet and said organic phase into a second inlet of a microfluidic cartridge thereby mixing said aqueous phase and said organic phase in a mixing device of the microfluidic cartridge to obtain an aqueous suspension of calibrated nanodroplets; and
d) Collecting the aqueous suspension of calibrated nanodroplets from the exit channel of the microfluidic cartridge.

2. The method according to claim 1, wherein said organic phase comprises an amphiphilic component and a low boiling fluorinated compound.

3. The method according to claim 1, wherein said low boiling fluorinated compound has a boiling point lower than 25° C.

4. The method according to claim 1, wherein said low boiling fluorinated compound is a low boiling perfluorocarbon.

5. The method according to claim 1, wherein said amphiphilic component comprises a phospholipid, and optionally a lipid bearing polymer.

6. The method according to claim 5, wherein said amphiphilic component further comprises a fatty acid.

7. The method according to claim 1, wherein step b) comprises the preparation of an organic phase comprising a low boiling fluorinated compound, said method comprising the steps of:
b.1) Condensing a gaseous low boiling fluorinated compound to obtain a liquid low boiling fluorinated compound;
b.2) Preparing a liquid organic solution comprising an organic solvent;
b.3) Cooling said liquid organic solution down to a temperature below the boiling point of said liquid low boiling fluorinated compound;
b.4) Mixing said liquid low boiling compound and said liquid organic solution to obtain an organic phase.

8. The method according to claim 7, wherein step b.1) is performed by cooling said gaseous low boiling fluorinated compound down to a temperature below the boiling point of said low boiling fluorinated compound.

9. The method according to claim 7, wherein at step b.4) the temperature at which the mixing is performed is a temperature below the boiling point of said low boiling fluorinated compound.

10. The method according to claim 7, wherein the temperature at step b.4) is the same temperature of step b.3).

11. The method according to claim 7, wherein the temperature of the obtained organic phase at the end of step b.4) is lower than the boiling point of said low boiling fluorinated compound.

12. The method according to claim 1, wherein step b) comprises the preparation of an organic phase comprising a low boiling fluorinated compound, said method comprising the steps of:
b.i) Preparing a liquid organic solution comprising an organic solvent, and
b.ii) Admixing a gaseous low boiling fluorinated compound to said organic liquid solution.

13. The method according to claim 12, wherein before the admixing of step b.ii) the temperature of the organic solution is above the boiling point of said gaseous low boiling fluorinated compound.

14. The method according to claim 13, wherein said temperature is between the boiling point and 25° C. higher than the boiling point.

15. The method according to claim 1, wherein said vesicles have a z-average diameter comprised between 100 nm and 1000 nm and a polydispersity lower than 0.20.

16. The method according to claim 7, wherein the temperature of the obtained organic phase at the end of step b.4) is at least 5° C. lower than the boiling point of said low boiling fluorinated compound.

17. The method according to claim 7, wherein the temperature of the obtained organic phase at the end of step b.4) is at least 10° C. lower than the boiling point of said low boiling fluorinated compound.

18. The method according to claim 7, wherein the temperature of the obtained organic phase at the end of step b.4) is at least 20° C. lower than the boiling point of said low boiling fluorinated compound.

19. The method according to claim 13, wherein said temperature is between the boiling point and 15° C. higher than the boiling point.

20. The method according to claim 13, wherein said temperature is between the boiling point and 10° C. higher than the boiling point.
